(19) United States
(12) Patent Application Publication
(10) Pub. No.: US 2015/0099987 A1
(43) Pub. Date: Apr. 9, 2015

(54) HEART RATE VARIABILITY EVALUATION FOR MENTAL STATE ANALYSIS

(71) Applicant: Affectiva, Inc., Waltham, MA (US)

(72) Inventors: Viprali Bhatkar, Cambridge, MA (US); Rana el Kaliouby, Milton, MA (US); Youssef Kashef, Obour City (EG); Ahmed Adel Osman, New Cairo (EG)

(21) Appl. No.: 14/569,691

(22) Filed: Dec. 13, 2014

Related U.S. Application Data
(63) Continuation-in-part of application No. 13/153,745, filed on Jun. 6, 2011.
Application No. 13/153,745 is a continuation-in-part of application No. 14/214,719, filed on Mar. 15, 2014.
Application No. 14/214,719 is a continuation-in-part of application No. 13/153,745, filed on Jun. 6, 2011.
(60) Provisional application No. 61/916,190, filed on Dec. 14, 2013.
(60) Provisional application No. 61/927,481, filed on Jan. 15, 2014.
(60) Provisional application No. 61/953,878, filed on Mar. 16, 2014.
(60) Provisional application No. 61/972,314, filed on Mar. 30, 2014.
(60) Provisional application No. 62/023,800, filed on Jul. 11, 2014.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 61/793,761, filed on Mar. 15, 2013.
(60) Provisional application No. 61/789,038, filed on Mar. 15, 2013.
(60) Provisional application No. 61/790,461, filed on Mar. 15, 2013.
(60) Provisional application No. 61/798,731, filed on Mar. 15, 2013.
(60) Provisional application No. 61/844,478, filed on Jul. 10, 2013.
(60) Provisional application No. 61/916,190, filed on Dec. 14, 2013.
(60) Provisional application No. 61/924,252, filed on Jan. 7, 2014.
(60) Provisional application No. 61/927,481, filed on Jan. 15, 2014.
(60) Provisional application No. 61/352,166, filed on Jun. 7, 2010.
(60) Provisional application No. 61/388,002, filed on Sep. 30, 2010.
(60) Provisional application No. 61/414,451, filed on Nov. 17, 2010.
(60) Provisional application No. 61/439,913, filed on Feb. 6, 2011.
(60) Provisional application No. 61/447,089, filed on Feb. 27, 2011.
(60) Provisional application No. 61/447,464, filed on Feb. 28, 2011.
(60) Provisional application No. 61/467,209, filed on Mar. 24, 2011.
(60) Provisional application No. 61/447,089, filed on Feb. 27, 2011.

(51) Int. Cl. A61B 5/16; A61B 5/00; A61B 5/024

(52) U.S. Cl. CPC A61B 5/165; A61B 5/02416; A61B 5/02405; A61B 5/7278; A61B 5/7246; A61B 5/6898; A61B 5/02438; A61B 5/0013; A61B 5/0022; A61B 5/6803
USPC 600/479

(57) ABSTRACT

A system and method for evaluating heart rate variability for mental state analysis is disclosed. Video of an individual is captured while the individual consumes and interacts with media. The video is analyzed to determine heart rate information with heart rate variability (HRV) being calculated and being understood to be in response to stimuli from the media. The analysis of heart rate variability is based upon a sympathovagal balance derived from a ratio of low frequency heart rate values to high frequency heart rate values. Heart rate variability is analyzed to determine changes in an individual's mental state related to the stimuli. Heart rate variability is determined and thereby mental state analysis is performed to evaluate media.

### RELATED APPLICATIONS

This application claims the benefit of U.S. provisional patent applications “Heart Rate Variability Evaluation for Mental State Analysis” Ser. No. 61/916,190, filed Dec. 14, 2013, “Mental State Analysis for Norm Generation” Ser. No. 61/927,481, filed Jan. 15, 2014, “Expression Analysis in Response to Mental State Express Request” Ser. No. 61/953,878, filed Mar. 16, 2014, “Background Analysis of Mental State Expressions” Ser. No. 61/972,314, filed Mar. 30, 2014, and “Mental State Event Definition Generation” Ser. No. 62/023,800, filed Jul. 11, 2014. This application is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. This application is also a continuation-in-part of U.S. patent application “Mental State Analysis Using Heart Rate Collection Based on Video Imagery” Ser. No. 14/214,719, filed Mar. 15, 2014 which claims the benefit of U.S. provisional patent applications “Mental State Analysis Using Heart Rate Collection Based on Video Imagery” Ser. No. 61/793,761, filed Mar. 15, 2013, “Mental State Analysis Using Blink Rate” Ser. No. 61/789,038, filed Mar. 15, 2013, “Mental State Data Tagging for Data Collected from Multiple Sources” Ser. No. 61/790,461, filed Mar. 15, 2013, “Mental State Well Being Monitoring” Ser. No. 61/798,731, filed Mar. 15, 2013, “Personal Emotional Profile Generation” Ser. No. 61/844,478, filed Jul. 10, 2013, “Heart Rate Variability Evaluation for Mental State Analysis” Ser. No. 61/916,190, filed Dec. 14, 2013, “Mental State Analysis Using an Application Programming Interface” Ser. No. 61/924,252, filed Jan. 7, 2014, and “Mental State Analysis for Norm Generation” Ser. No. 61/927,481, filed Jan. 15, 2014 that is also a continuation-in-part of U.S. patent application “Mental State Analysis Using Web Services” Ser. No. 13/153,745, filed Jun. 6, 2011, which claims the benefit of U.S. provisional patent applications “Mental State Analysis Through Web Based Indexing” Ser. No. 61/352,166, filed Jun. 7, 2010, “Measuring Affective Data for Web-Enabled Applications” Ser. No. 61/388,002, filed Sep. 30, 2010, “Sharing Affect Data Across a Social Network” Ser. No. 61/414,451, filed Nov. 17, 2010, “Using Affect Within a Gaming Context” Ser. No. 61/439,913, filed Feb. 6, 2011, “Recommendation and Visualization of Affect Responses to Videos” Ser. No. 61/447,089, filed Feb. 27, 2011, “Video Ranking Based on Affect” Ser. No. 61/447,464, filed Feb. 28, 2011, and “Baseline Face Analysis” Ser. No. 61/467,209, filed Mar. 24, 2011. The foregoing applications are each hereby incorporated by reference in their entirety.

### FIELD OF ART

This application relates generally to heart rate analysis and more particularly to heart rate variability evaluation for mental state analysis.

### BACKGROUND

An individual's emotions are an important component of who the individual is, and each individual experiences numerous emotional states on a regular basis. Mental states are experienced in response to everyday events such as frustration during a traffic jam, boredom while standing in line, and impatience while waiting for a cup of coffee. Human emotion manifests as a potent mix of an individual's mood and attitude and, when considered in context, can be used to provide valuable insight into customer experience in a retail setting or e-commerce application, for instance. Analyzing the mental states of people can help to interpret individual or collective responses to surrounding stimuli. The stimuli can range from watching videos and sporting events to playing video games, interacting with websites, and observing advertisements, to name a few. There are a growing number of emerging applications which can benefit from the capability of detecting human emotion. Applications for automated human emotion detection include education, training, speech therapy, and analysis of media content to name a few.

Determining a person's mental state is a difficult task. Often, the underlying feelings of people are subliminal and unarticulated, thus rendering the mood, thoughts, or mental state of people complex to ascertain. However, even when left unarticulated, mental states often affect how a person behaves and interacts with others on a given day. Given this reality, a wide variety of methods are used across a spectrum of applications to help understand a person's mental state. A wide variety of techniques must be employed, given that the complex detection and interpretation of facial expressions under varying conditions is a task humans intuitively and constantly perform. Various instruments and methods have been developed for observing human emotions in psychology, the neurosciences, and machine learning studies. Sensors can detect emotional cues by directly measuring physiological data, such as skin temperature and galvanic resistance. Additionally, different methods for determining mental state analysis need not exist on their own. Analysis from multiple methods for evaluating a person's mental state combined and cross checked with one another can provide a more accurate assessment of the emotional state of an individual than is possible considering only one source.

### SUMMARY

The variability of an individual's heart rate can be evaluated to analyze the mental state of the individual. Heart rate evaluation is used to correlate an event or moment to a human emotional response, which is useful in assessing the effectiveness of media content such as advertising, editorials, documentaries, and the like. A computer-implemented method for mental state analysis is disclosed, comprising: obtaining video of an individual; analyzing the video to determine heart rate information; calculating a heart rate variability value (HRV) based on the heart rate information; and evaluating a physiological arousal based on the HRV. The calculating of heart rate variability can be performed using a sliding time window. The method can include determining the context during which the heart rate information is captured.

Various features, aspects, and advantages of various embodiments will become more apparent from the following further description.

### BRIEF DESCRIPTION OF THE DRAWINGS

The following detailed description of certain embodiments may be understood by reference to the following figures wherein:

FIG. 1 is a flow for heart rate variability analysis usage.

FIG. 2 shows example heart rate components.

FIG. 3 shows example arousal observed in time windows.

FIG. 4 shows example arousal with varying width windows.

FIG. 5 shows example arousal summary metrics.

FIG. 6 shows example degrees of emotional content.

FIG. 7 is a diagram showing various image collection devices.

FIG. 8 is a flow for physiology analysis.

FIG. 9 is a flow for physiology analysis with video.

FIG. 10 is a flow for physiology analysis with a server.

FIG. 11 is an example system for heart rate variability analysis usage.

### DETAILED DESCRIPTION

The heart can provide a vast amount of information about a human body because heart rate constantly adjusts from beat to beat to meet the demands of everyday life. Heart rate variability (HRV), a measure of the magnitude and timing of changes in heart rate, can be evaluated to provide insight into the mental state of an individual. Heart rate variability as a value reflects the beat-to-beat changes in heart rate. Heart rate is typically controlled by the autonomic nervous system (ANS), the part of the nervous system that acts as a control system for largely involuntary human functions such as respiratory rate, digestion, pupil dilation, perspiration, and heart rate. Here, it is important to note that most ANS controlled functions are involuntary, but a number of ANS actions can change or engage based on a certain degree of conscious control, such as breathing or swallowing. At the core of the human body, the beating heart is constantly being acted upon by the ANS through two different branches of control, with heart rate being regulated constantly though balanced input from the two components. On one side, the sympathetic nervous system expends energy and acts on the heart in the case of emergencies that cause stress and that provoke the so called “fight or flight” response. In such a situation, the sympathetic nervous system activates an increase in heart rate (among other actions) to deal with increased blood requirements from excited muscles. On the other hand, the parasympathetic, or vagal, nervous system acts on the heart to conserve energy in non-emergency situations, so called “rest and digest” scenarios, by activating a decrease in heart rate that allows blood flow to normalize and returns blood to areas such as the stomach for food processing.

The disclosed concepts provide methods of measuring attention and arousal in response to stimuli, such as an advertisement, by using an evaluation of heart rate variability. The evaluation of heart rate variability includes an analysis of heart rate and other information, including the context under which an individual experiences stimuli. Computer analysis is performed on facial and/or heart rate information to determine the mental states of viewers as they observe various types of stimuli. A mental state can be a cognitive state, an emotional state, or a combination thereof. Examples of emotional states include happiness and sadness, while examples of cognitive states include concentration and confusion. Observing, capturing, and analyzing mental states such as these can yield significant information about viewers' reactions to various stimuli. In embodiments, the mental state data is rendered on a computer display. In other embodiments, the mental state data is stored for later analysis and/or transmitted to a mobile platform. In embodiments, the mental state data is transmitted to a server. In other embodiments, mental state data received from a server is used to render mental state information via audio, via a display, or via both audio and a display.

Analysis of heart rate variability can include identifying a location of a face or a set of faces of an individual or multiple individuals in a portion of a video. Facial detection can be performed using a facial landmark tracker. In embodiments, the tracker identifies points on a face and is used to locate sub-facial parts such as the forehead and/or cheeks. Further, skin detection can be performed and facial portions can be removed from images where the portions are considered irrelevant. In some cases eyes, lips, or other portions which have been deemed irrelevant can be ignored within images. The method can further comprise establishing a region of interest (ROI) including the face or a portion thereof. In at least one embodiment, the ROI can be defined as a portion of a box returned as the location of the face, such as the middle 60% of the width of the box and the full height of the box. In another embodiment the ROI can be obtained via skin-tone detection and can be accomplished using various regions of skin on an individual's body, including non-facial regions. In some embodiments the ROI can be processed using various image processing techniques including, but not limited to, sharpness filters, noise filters, convolutions, and brightness and/or contrast normalization that can, individually or in concert, operate on a single frame or a group of frames over time. In embodiments the method is able to scale its analysis to process multiple faces within multiple regions of interests (ROI) returned by the facial landmark detector.

The method can further comprise separating temporal pixel intensity traces in the regions of interest into at least two channel values and spatially and/or temporally processing the separated pixels to form raw traces. While one embodiment establishes red, green and blue as channel values, other embodiments can base channels on other color choices or other functions of the pixel intensity traces. The channels of the video can be analyzed on a frame-by-frame basis and spatially averaged to provide a single value for each frame in each channel. Some embodiments use a weighted average to emphasize certain areas of the face. One raw trace per channel can be created and can include a single value that varies over time. Additionally, the raw traces can be processed for filtering or enhancement. Such processing can include various filters such as low-pass, high-pass, or band-pass filters; interpolation; decimation; or other signal processing techniques. In at least one embodiment, the raw traces are de-trended using a procedure based on a smoothness-priors approach. Alternatively, other types of analysis are possible, such as a feature being extracted from a channel based on a discrete probability distribution of pixel intensities. A histogram of intensities can be generated with a single histogram per channel. In some embodiments, one bin can be considered equivalent to a spatial summation. Analysis can include tracing fluctuations in reflected light from the skin of a person being viewed.

The method can further comprise decomposing the raw traces into at least one independent source signal. The decomposition can be accomplished using independent component analysis (ICA). Independent component analysis (ICA) is a technique for uncovering independent signals from a set of observations composed of linear mixtures of the underlying sources. In this case, the underlying source signal of interest can be blood volume pulse (BVP). To explain further, during the human cardiac cycle, volumetric changes in blood vessels close to the skin of an individual modify the path length of any incident ambient light striking the skin, which in turn changes the amount of light reflected from the skin, an observation which can make possible the timing of cardiovascular events by measuring reflected light on the skin of an individual. By capturing a sequence of images of the facial region with a webcam, the red, green and blue (RGB) color sensors pick up a mixture of reflected plethysmographic signals along with other sources of fluctuations in light due to artifacts. Given that hemoglobin absorptivity differs across the visible and near-infrared spectral range, each color sensor records a mixture of the original source signals with slightly different weights. The ICA model assumes that the observed signals are linear mixtures of the sources where one of the sources can be hemoglobin absorptivity or reflectivity. The ICA model can be used to decompose the raw traces into a source signal representing hemoglobin absorptivity correlating to BVP. Respiration rate information is also determined in some embodiments.

The method can further comprise processing at least one source signal to obtain the heart rate information. Heart rate (HR) can be determined by observing the intervals between peaks of the source signal. That is, using the information obtained from the color sensors and assuming that differences in reflected light can correspond to differences in the hemoglobin content of blood (e.g. recently oxygenated blood from the heart having a higher hemoglobin load), the time interval between bursts of oxygenated blood can be calculated. Thus, the heart rate information can include heart rate, and the heart rate can be determined based on changes in the amount of reflected light. Heart rate variability, both phasic and tonic, can be obtained using a power spectral density (PSD) estimation and/or through other signal processing techniques. The analysis can include evaluation of phasic and tonic heart rate responses. In some embodiments, the video includes a plurality of other people. Such embodiments can comprise identifying locations for faces of the plurality of other people and analyzing the video to determine heart rate information on the plurality of other people. Heart rate variability can be determined based on the heart rate information collected.

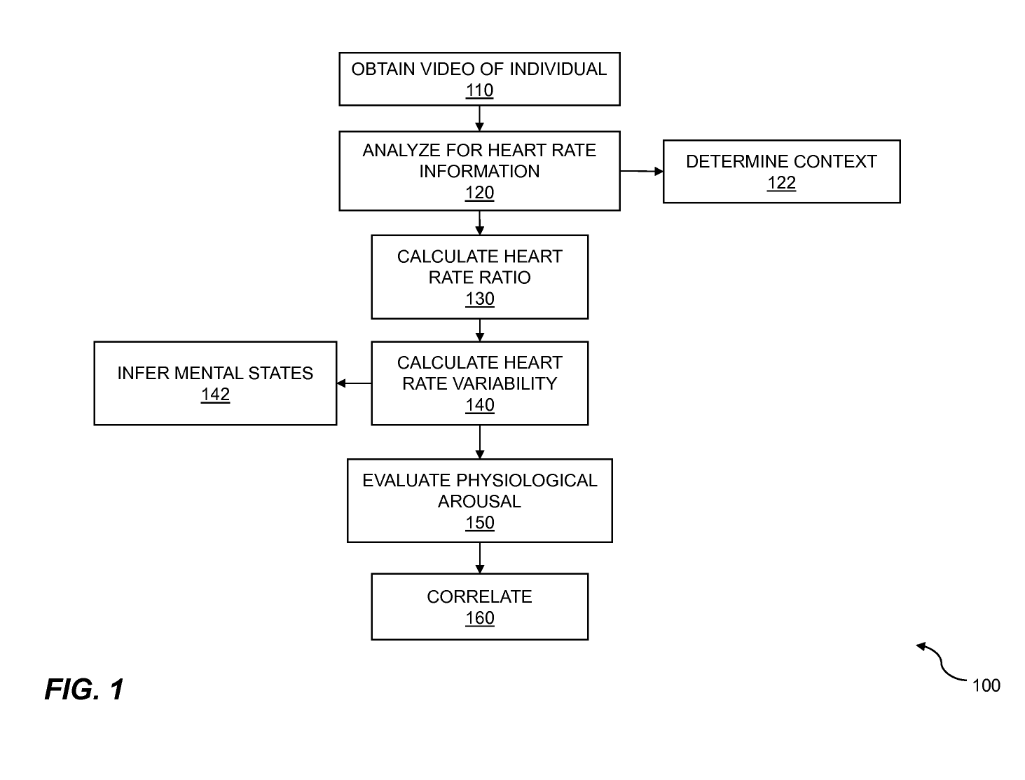

FIG. 1 is a flow for heart rate variability analysis usage. The flow 100 describes a computer-implemented method for mental state analysis. The flow 100 includes obtaining video of an individual 110. The video can be analyzed to collect mental state data for the individual. The collecting of mental state data from the video can include collecting action units, facial expressions, and the like. The collecting of mental state data from the video can also include collecting data consisting of the pulse wave generated by the human heart. The pulse wave is initiated by a heartbeat and travels through the whole vascular system, beginning with the major arteries and reaching the face through the carotid artery, where it causes a short-term change in blood volume. The heart information can be collected from video of a person. The heart information can be augmented by a biosensor attached to a person. The biosensor can be attached to various portions of a person including, for example, a finger.

The flow 100 includes analyzing the video to determine heart rate information 120. The analysis can include an evaluation of data including the heart's pulse wave. The evaluation can also include an analysis of skin color change on an individual as a function of changes in blood volume. Other methods to extract heart rate information can also be applied when analyzing a video to determine heart rate information. In certain embodiments, the analysis includes determining the context 122, circumstances, and setting under which the video of the individual was recorded. The context can include the circumstances surrounding the watching of a video or advertisement, the playing of a game, the interaction of an individual with elements of a web page, or other examples of auxiliary information which can give a fuller picture of the recording of the video and the surrounding context.

The flow 100 includes calculating the heart rate ratio 130 based upon the heart rate information. The heart rate ratio can be influenced by an increase in heart rate driven by the sympathetic nervous system. The heart rate ratio can also be influenced by a decrease in heart rate driven by the parasympathetic or vagal nervous system. Calculating the ratio can include implementing the concept of sympathovagal balance (SB), which involves the study of the often-reciprocal actions of the sympathetic and vagal nerve outflows on the human heart. The heart rate ratio can be expressed at a single point in time. The heart rate ratio can also be expressed as an average or range over a period of time.

The flow 100 includes calculating a heart rate variability value 140 based on the heart rate information. Heart rate variability is a measure of beat-to-beat (or of an inter-beat interval) changes in heart rate. The changes occur naturally in all people and can be analyzed as an aid in determining an individual's overall wellbeing. Heart rate variability can be measured in time domain values. The measured time domain intervals can represent the heart beat as a wave, as it is displayed on an electrocardiogram machine. Heart rate variability can also be measured in frequency domain measures. Typically, frequency domain measures provide a spectral analysis value of heart rate. A spectral analysis of heart rate variability isolates the signals coming from the human heart using different frequency measurements in order to arrive at maximum independence for each of the portions of the signal primarily influenced by sympathetic or parasympathetic nervous system components. In embodiments, either or both of the frequency domain measures and time domain measures can be calculated. The heart rate variability value can include both the sympathetic nervous system value and the parasympathetic nervous system value of the autonomic nervous system. The heart rate variability can be based on sympathovagal balance where the sympathovagal balance is determined based on a ratio of a low frequency heart rate sympathetic value to a high frequency heart rate parasympathetic value.

The flow 100 can further comprise inferring mental states 142 based on the heart rate variability. The inference can be a type of mental state data. For example, a heart rate variability measurement can infer a mental state of high arousal or interest. Heart rate variability can be analyzed to infer mental states such stress, sadness, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, and curiosity.

The flow 100 includes evaluating a physiological arousal 150 based on the heart rate variability. Arousal can range from being highly activated, such as when someone is agitated, to being entirely passive, such as when someone is bored. The flow 100 can include evaluating valence. Valence represents a way to measure the intrinsic attractiveness or averseness of an event or situation. Valence can range from being very positive, such as when someone is happy, to being very negative, such as when someone is angry. The valence can be a function of a media presentation which an individual or group of people are viewing or with which they are interacting. The valence can be used to analyze the media.

The flow 100 further comprises correlating 160 the heart rate ratio to the physiological arousal. In some cases, the arousal can cause an increase in the heart rate ratio. In other cases, the arousal can cause a decrease in the heart rate ratio. The physiological arousal can be used in media analysis. By detecting arousal based upon heart rate variability while considering the media being observed at the time of the arousal detection, the mental state of an individual can be usefully correlated to events within a piece of media content. In some cases, a variation in heart rate can be correlated to a video or other media that a viewer is experiencing. In some embodiments, it is possible to determine whether the heart rate variability is in response to the media being viewed. The physiological arousal can include an emotional response. The viewer's emotional reaction to the media can be inferred based on the heart rate variability. Further, the media can then be rated based on the arousal.

The physiological arousal can be factored into well-being status evaluation. A computer can be used to collect mental state data from an individual, including physiological arousal data; analyze the mental state data; and render an output that provides the well-being status of the individual. The well-being status can then be presented to the individual as feedback which can include recommending activities, eliminating activities, and identifying a potentially impaired state. A well-being status evaluation can also be performed by a computer-implemented method for mental state analysis including receiving a well-being status based on mental state data obtained on an individual wherein the well-being status results from analyzing the physiological arousal. The well-being status can then be rendered as an output. The well-being status can be correlated to media that is being viewed. Various steps in the flow 100 may be changed in order, repeated, omitted, or the like without departing from the disclosed concepts. Various embodiments of the flow 100 may be included in a computer program product embodied in a non-transitory computer readable medium that includes code executable by one or more processors.

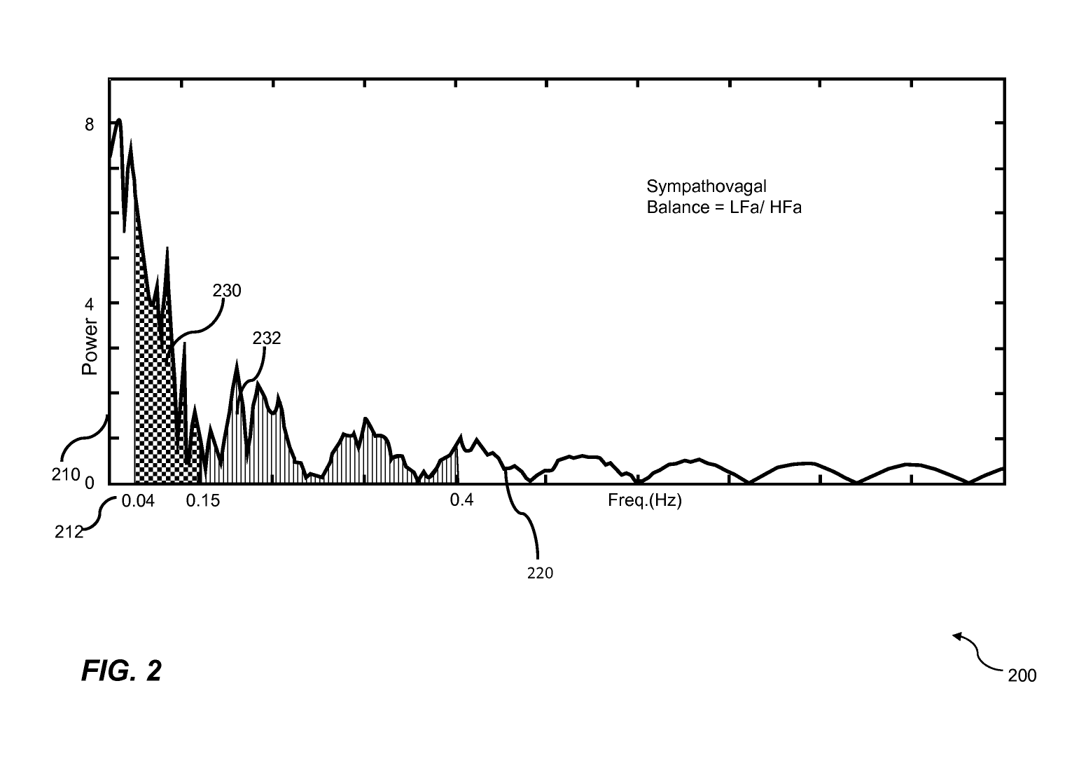

FIG. 2 shows example heart rate components. The diagram 200 shows a graph with an example of heart rate variability measured in frequency domain measures. Typically, frequency domain measures provide a spectral analysis value of heart rate. The Y-axis of the graph measures the power 210, and can also be considered the power spectral density. The strength of the frequency can thus be plotted on the graph. The X-axis of the graph measures the frequency 212 in hertz. A measurement 220 can be plotted on the graph that represents heart rate variability. The heart rate variability can be considered to have two fundamental oscillatory components with the first being a low frequency band 230 ranging between 0.04 and 0.15 Hz, which is driven by the combined factors of vagal and sympathetic components. The sympathetic nervous system can aid in causing a fight-or-flight response and can activate an increase in heart rate. The second component includes a high-frequency band 232 ranging from 0.15 to 0.4 Hz. This component can show the effects of the parasympathetic nervous system including respiratory modulation and the value of the high-frequency band component can indicate the magnitude of the influence of the vagus nerve on the heart, an influence which can provoke the so-called “rest-and-digest” response. The vagus nerve increases activity in non-emergencies and allows humans to rest. The vagal nervous system can enable a decrease in heart rate.

Therefore, it can be seen that a spectral analysis of heart rate variability is comprised of a sympathetic band and a parasympathetic band. The heart rate variability can be determined based on a low frequency heart rate sympathetic value determined from the heart rate information. The low frequency heart rate value can be based on measurements between 0.04 Hz to 0.15 Hz. The heart rate variability can be determined based on a high frequency heart rate value determined from the heart rate information. The high frequency heart rate value can be based on measurements between 0.15 Hz to 0.4 Hz. The example 200 further comprises calculating a heart rate ratio based on the low frequency heart rate value and the high frequency heart rate value. In embodiments, the physiological arousal identified by the measured heart rate variability can be correlated to the heart rate ratio.

The heart rate variability can also be based on sympathovagal balance. Sympathovagal balance is determined from the notion that sympathetic and parasympathetic influences can be measured and represented in a single value using a formula of low frequency and high frequency heart measurements. In normal subjects, periods of sympathetic and parasympathetic dominance fluctuate throughout the day. The formula takes into account that low frequency heart variations that are a mix of both sympathovagal and parasympathovagal influences, and that high frequency heart variations include only parasympathovagal influences.

Another embodiment for heart rate variability determination can be based on an estimate of a blood volume pulse (BVP) signal obtained from a person. A heart rate (HR) can be estimated from the BVP, and heart rate variability can be determined from the estimated BVP values over time. The heart rate of a person can be monitored with a camera by using noninvasive photo-plethysmography (PPG) techniques. The PPG techniques can measure a cardiovascular BVP signal by analyzing properties of light reflected from the skin of the person. Since blood can absorb more light than surrounding tissue, variations in blood volume can cause variations in the amount of light reflected from the face of the person. The estimation of the BVP can be based on a supervised machine learning technique. The face of the person can be monitored using ambient light and a camera, where the camera can be a variety of types of image capture devices that can include a webcam, a video camera, a room camera, a still camera, a thermal imager, a CCD device, a smartphone camera, a three-dimensional camera, a light field camera, multiple cameras to obtain different aspects or views of a person, or any other type of image capture technique to capture data to be used in an electronic system. The data received from the camera that monitors the face of the person can be analyzed and used to estimate the blood volume pulse. The estimation of the BVP can be determined by training a discriminative statistical model.

The signal obtained from the camera used to monitor the person contains BVP signals and noise signals. Based an understanding that changes in the intensity of light reflected from the face of the person are a function of a gush of blood flow that have unique characteristics differing from noise characteristics, the blood flow signals can be differentiated from the noise signals. That is, the unique BVP signal characteristics can be learned empirically from the data. The face of the person can be localized using a face tracker. In the case that the camera used to monitor the person can produce red-green-blue (RGB) signals, the mean of the green channel can be extracted from a face region of interest (ROI) of the person. A feature at time t can be computed as the spatial average over the green channel ROI. A BVP “ground truth” signal can be aligned with features extracted from the mean of the green channel. The alignment can be used to train a classifier to learn a one-to-one mapping between the BVP ground truth signal and the mean of the green channel. For example, the alignment can include aligning peaks of the ground truth BVP signal (maximum blood flow) with minima of the mean of the green signal (minimal reflection of light). A feature representation can be based on a temporal representation of the mean of the green channel. A feature at time t can correspond to extracting a window of size wₛ seconds centered at t from the mean of the green channel. Features that can be used from training and testing are extracted at minima timestamps and can be half way between each two successive minima timestamps of the mean of the green channel. The discriminative model used can be a support vector machine (SVM) that can be with a radial basis function (RBF) kernel. The SVM can be trained and can be used as a sliding window. The timestamps of local peaks in a response of the SVM model can be potential timestamps of heart beat locations. The magnitude of the classifier output can be used as a confidence metric to filter out potentially false beats introduced by noise. A conservative HR signal can be computed from a raw mean of the green channel when HR estimates deviate significantly from a moving average. A confidence score can be measured by calculating a percentage of a number of confident heartbeats from a total of beats that can be detected over a period of time.

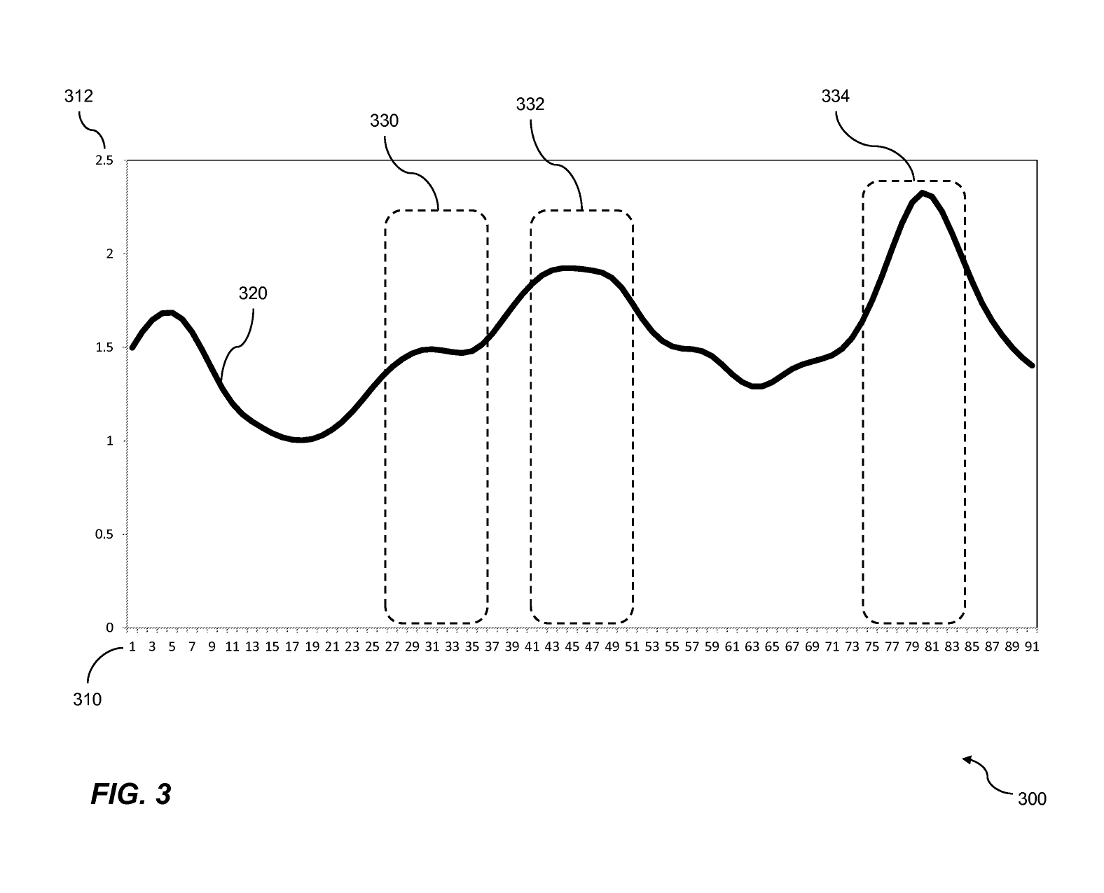

FIG. 3 shows example arousal observed in time windows. The diagram 300 represents a graph that depicts arousal measured over time. The physiological arousal can be used in media analysis where multiple media instances have heart rate variability values that are correlated to individuals' emotional response for the multiple media instances. The X-axis 310 of the diagram measures the time period of a media example in seconds. The Y-axis 312 of the diagram shows the sympathovagal value. The values plotted can be sympathovagal balance values for a 30-second period of time or some other duration. The values can be summed or averaged over the period of time or in some other way combined. The sympathovagal value can be that of an individual or it can be an average for a plurality of individuals. Heart rate variability (HRV) based upon sympathovagal balance values 320 can be plotted as an individual is experiencing a media instance, such as an advertisement, web site, game, television, movie or other media. Analysis can include evaluation of area under the curve (AUC) for sympathovagal balance values.

Physiological arousal can be measured in various time windows including, for example, the time windows 330, 332, and 334. Time segments can be identified that exhibit various states of arousal and are correlated to the media. Within these time segments, levels of individual arousal can be associated with the messages in the media. Levels of arousal can also be compared to each other to analyze the emotional effect of messages in different time windows. For example, advertisements or other media with high emotional content can result in higher levels of arousal. In some embodiments, the analysis can be performed on a continuous basis over time. In other embodiments, calculations of arousal measurements are performed on a sliding window model.

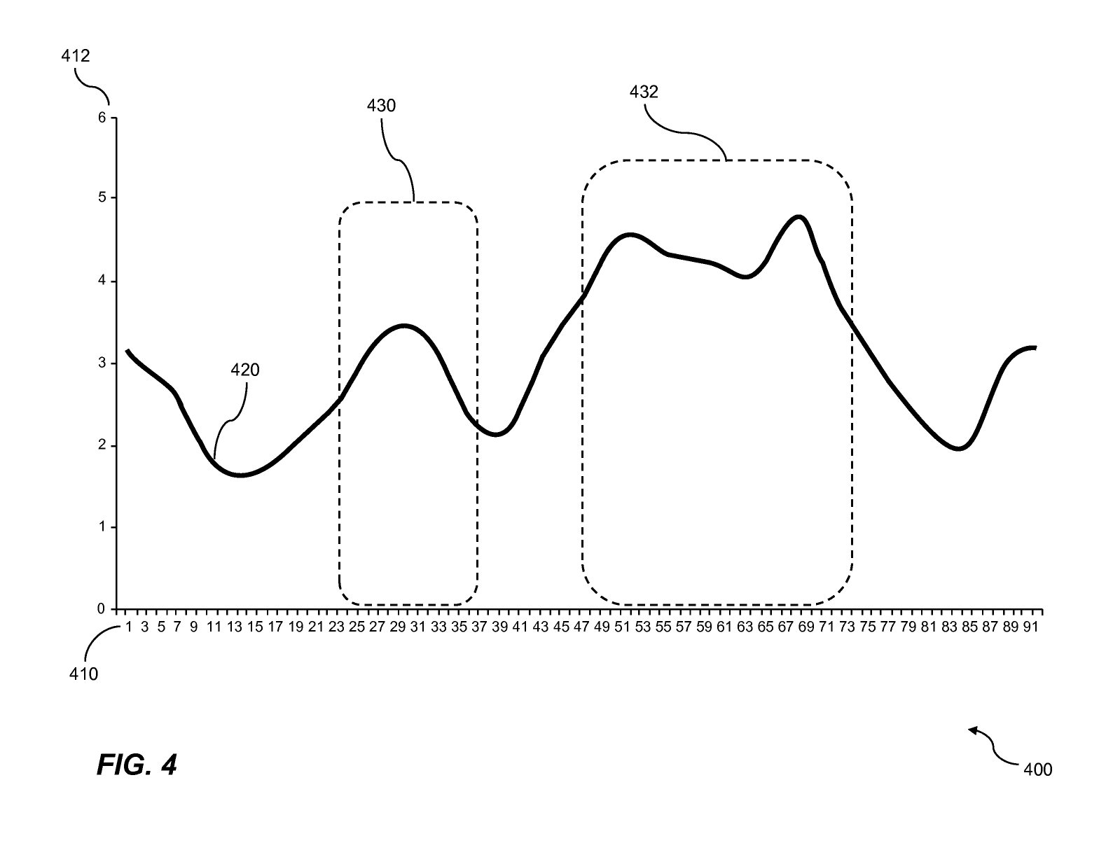

FIG. 4 shows example arousal with varying width windows. The diagram 400 represents a graph that depicts arousal measured over time. The X axis 410 of the diagram shows the time period of a media example in seconds. The Y axis 412 of the graph shows the sympathovagal value. Heart rate variability based upon sympathovagal balance values 420 can be plotted as an individual is experiencing a media instance, such as an advertisement, web site, game, television, movie or other media.

Physiological arousal can be measured in various time windows including, for example, time windows 430 and 432. Time segments can be identified that exhibit various states of arousal and those segments can be correlated to the media. Within these time segments, levels of arousal can be correlated to the messages in the media. Levels of arousal can also be compared to each other to analyze the emotional effect of messages in different time windows. In some embodiments, the analysis can be performed on a continuous basis. In other embodiments, the analysis can be for values averaged over a period of time, such as 30 seconds or some other period of time. In some cases, the time window can vary in duration so that certain events are evaluated with a finer granularity of time. In embodiments, calculations of arousal measurements are performed using a sliding window model.

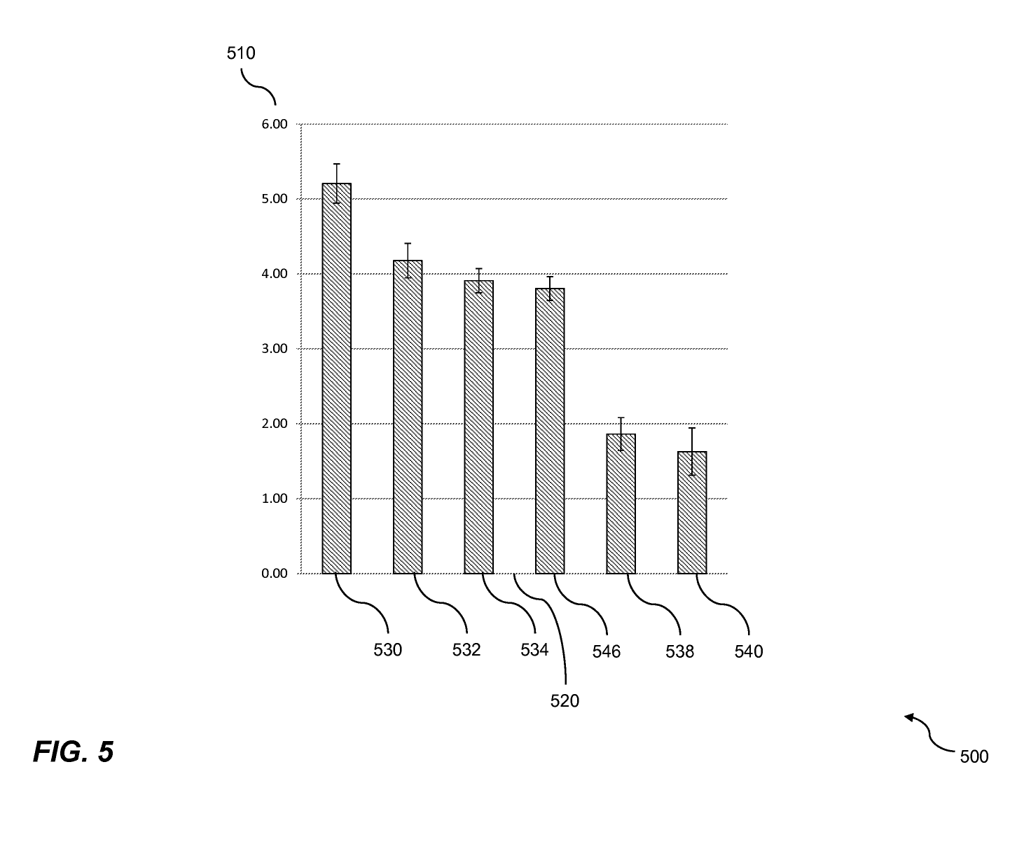

FIG. 5 shows an example of arousal summary metrics. The diagram 500 depicts a graph that displays physiological arousal values for different media content. The quantified physiological arousal can be used in media analysis. The Y-axis 510 of the graph shows the sympathovagal balance value. The sympathovagal balance value can be for an individual or it can be an average taken for a plurality of individuals. The X-axis 520 of the graph displays the different media being analyzed. Sympathovagal balance values on the Y-axis 510 can be plotted as an individual is experiencing a media instance, such as an advertisement, web site, game, television, movie, or other media. As depicted in the graph diagram 500, a media instance 530 such as an advertisement can have a high sympathovagal value, which can correlate to media with highly emotional content. Another media instance 532, such as an advertisement, with a smaller sympathovagal value can correlate to media with less emotional content. Other media 534, 536, 538, 540 with various sympathovagal values can correlate to media with varying levels of emotional content. Thus the diagram 500 demonstrates that sympathovagal values, derived from an analysis of heart rate variability, can be used to provide summary metrics that correlate arousal and mental state to emotional content and can be used to analyze and compare different media content.

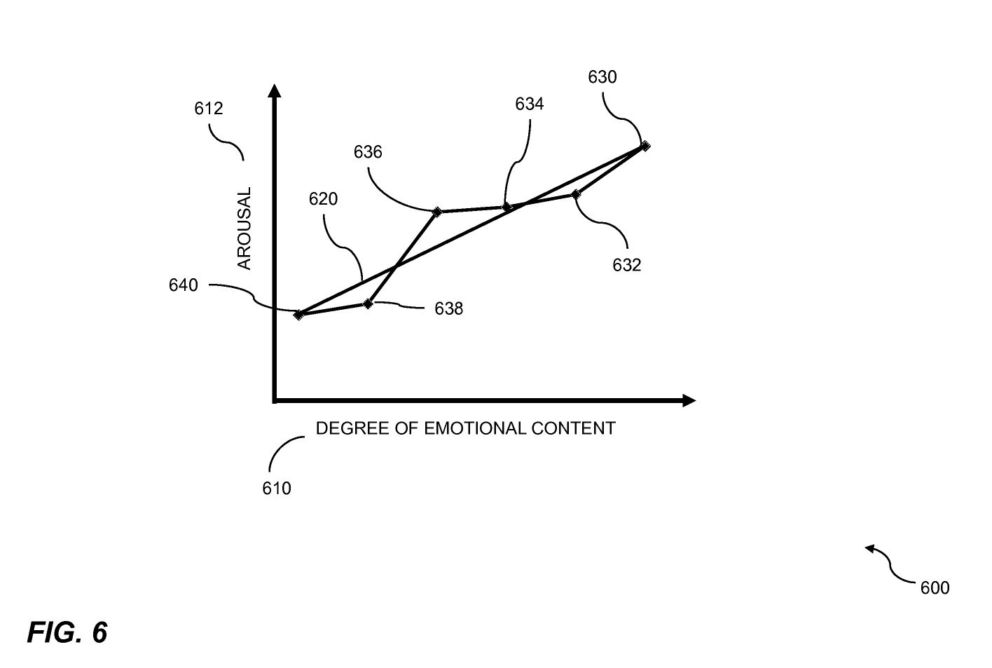

FIG. 6 shows example degrees of emotional content. The physiological arousal can be driven by an emotional response. The diagram 600 presents a graph that displays the level of arousal for a media instance, such as an advertisement, and correlation between the arousal and the degree of emotional content in the media. The physiological arousal can be used in media analysis where multiple media instances have heart rate variability values that are correlated to the emotional response for the multiple media instances. The X-axis 610 of the diagram shows the degree of emotional content present in the media. The Y-axis 612 of the graph measures the level of arousal based upon the heart rate variability, which in turn is used to determine the sympathovagal value. In the diagram, media instances are plotted at an X-Y coordinate according to the level of arousal and degree of emotional content contained in the instance. Referring to the media examples of FIG. 5, the media with the highest sympathovagal value, the first instance 530 from FIG. 5, is plotted highest and furthest to the right 630 in the current graph 600 due to the instance's high degree of emotional content. Referring again to the media examples of FIG. 5, the media with the lowest sympathovagal value, the final instance 540 from FIG. 5, is plotted lowest and furthest to the left 640 in the current graph 600 due to the instance's low degree of emotional content. Other media instances from FIG. 5 532, 534, 536, 538, 540 are likewise plotted on the present graph 600 using points 632, 634, 636, 638, 640 to show the instances' relative sympathovagal values and degrees of emotional content.

Thus the diagram 600 demonstrates that levels of arousal can be correlated to the degree of emotional content in a media instance. Levels of arousal can be compared to each other to analyze the emotional effect of messages with different levels of emotional content. A linear analysis 620 can be displayed to depict the relationship between the increased degree of emotional content and the increase in sympathovagal value.

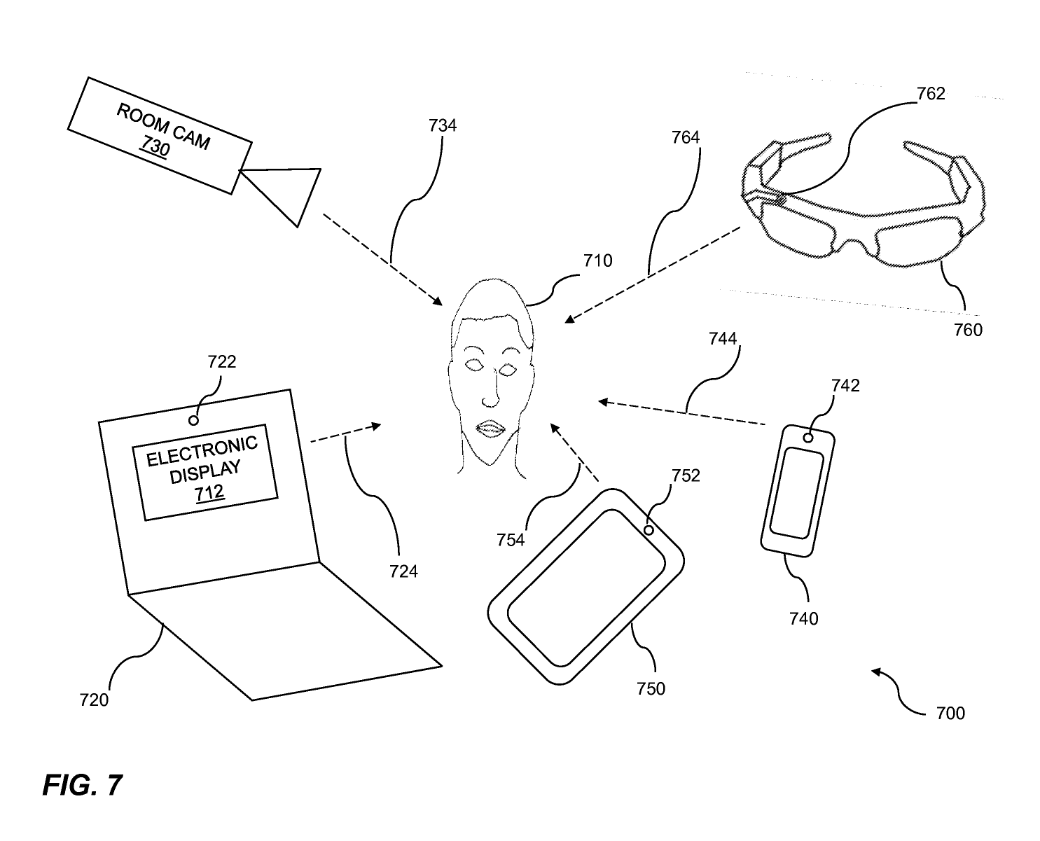

FIG. 7 is a diagram 700 showing various image collection devices. The collection of image and video data is used to capture heart rate information and other information that can be used to determine levels of arousal and/or response to degrees of emotional content. A user 710 can be performing a task, such as viewing a media presentation on an electronic display 712 or doing another task where it might be useful to determine the user's mental state. The heart rate information can be gathered while the individual views a collection of digital media. The collection of digital media can comprise one or more of a movie, a television show, a web series, a webisode, a video, a video clip, an electronic game, an e-book, or an e-magazine.

The electronic display 712 can be on a laptop computer 720 as shown, a tablet computer 750, a cell phone 740, a desktop computer monitor, a television, or any other type of electronic device. The heart rate information can be collected on a mobile device such as a cell phone 740, a tablet computer 750, or a laptop computer 720, and can be collected through a biosensor, which can be wearable. The heart rate information can be obtained from multiple sources and can be augmented by one or more biosensors. Thus, the multiple sources can include a mobile device, such as a phone 740, a tablet 750, or a wearable device such as glasses 760. A mobile device can include a forward facing camera and/or a rear facing camera that can be used to collect mental state data. Facial data can be collected from one or more of a webcam 722, a phone camera 742, a tablet camera 752, a wearable camera 762, and a room camera 730. The analyzing of the mental state data can be accomplished, at least in part, on a device doing the collecting of the mental state data.

As the user 710 is monitored, the user 710 can exhibit heart rate variability due to the nature of the task, boredom, distractions, or for other reasons. As the user moves, the user's face might be visible from one or more of the multiple sources. Thus, if the user 710 is looking in a first direction, the line of sight 724 from the webcam 722 might be able to observe the individual's face, but if the user is looking in a second direction, the line of sight 734 from the room camera 730 might be able to observe the individual's face. Further, if the user is looking in a third direction, the line of sight 744 from the phone camera 742 might be able to observe the individual's face. If the user is looking in a fourth direction, the line of sight 754 from the tablet camera 752 might be able to observe the individual's face. If the user is looking in a fifth direction, the line of sight 764 from the wearable camera 762 might be able to observe the individual's face. Thus, the collection of mental state data and/or heart rate information can occur through a single image capturing device or multiple image capturing devices. In embodiments, the collection of mental state data and/or heart rate information is collected sporadically.

A wearable device such as the pair of glasses 760 as shown can be worn by another user or an observer. In some embodiments, the wearable device is a device other than glasses, such as an earpiece with a camera, a helmet or hat with a camera, a clip-on camera attached to clothing, or any other type of wearable device with a camera or other sensor for collecting mental state data. The individual 710 can also wear a wearable device including a camera which can be used for gathering contextual information and/or collecting heart rate information on other users. Because the individual 710 can move his or her head, the facial data can be collected intermittently when the individual is looking in a direction of a camera. At times, multiple cameras can observe a single person. In some cases, multiple people can be included in the view from one or more cameras, and some embodiments include filtering out faces of one or more other people to determine whether the individual 710 is looking toward a camera.

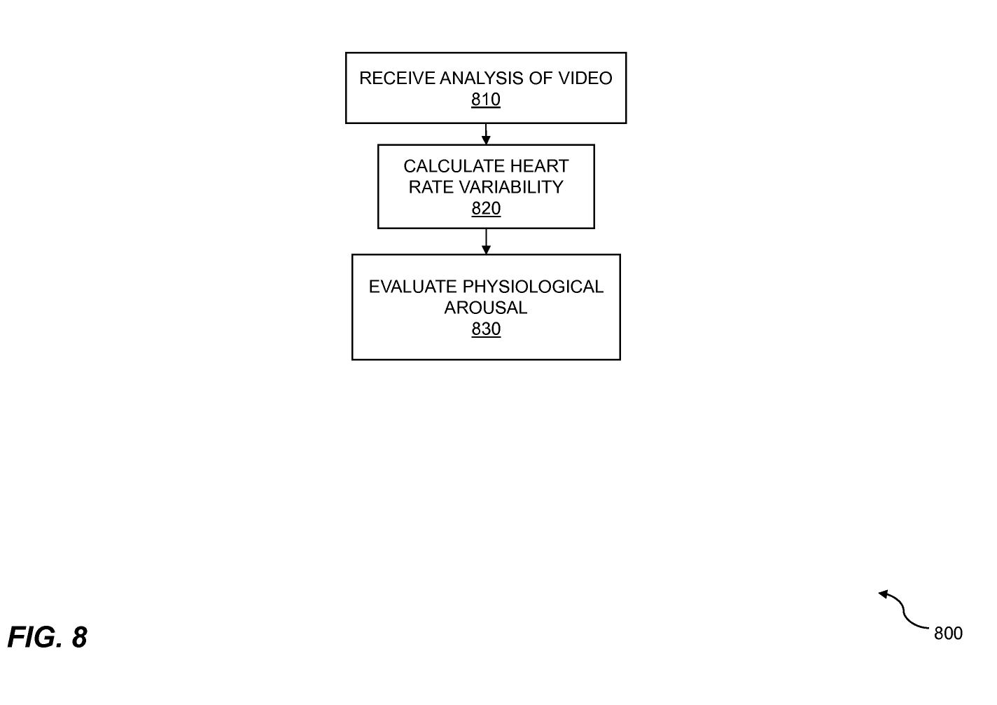

FIG. 8 is a flow for physiology analysis. The flow 800 describes a computer-implemented method for physiology analysis associated with heart rate variability evaluation. This flow 800 can be considered to be from a server perspective. The flow 800 can include receiving analysis of a video 810 to determine heart rate information. The analysis information can include both video and collected information relating to mental states. The video analysis can also infer a heart rate variability. The video analysis can include an algorithm that detects the underlying heartbeat. The video analysis can include measurements of differences in the reflectivity of ambient light off an individual's face that change over time in order to detect and display variations in heart rate. Other information or methods of analyzing and determining heart rate variability can be presented in the video analysis. The video analysis can include other information about the mental state of the individual, including facial data, physiological data, accelerometer data, and analysis of an individual's face to extract and interpret laughs, smiles, frowns, and other facial expressions.

The flow 800 can include calculating a heart rate variability value 820 based on the heart rate information. Sympathetic and parasympathetic components of the heart rate can be used individually or in combination to arrive at a heart rate variability value. The heart rate variability value can be based upon the heart rate ratio. The heart rate information used to create the value can include mental state data including data from facial analysis, biosensors, or physiological data.

The flow 800 can include evaluating physiological arousal 830 based on the heart rate variability value. Mental state data including data from facial analysis, biosensors, or physiological data can be used to augment the heart rate information. The evaluation can include an assessment of the degree of emotional content associated with the video analysis. The evaluation can include the context under which the evaluation is made. The evaluation can be undertaken for a point in time or over a period of time.

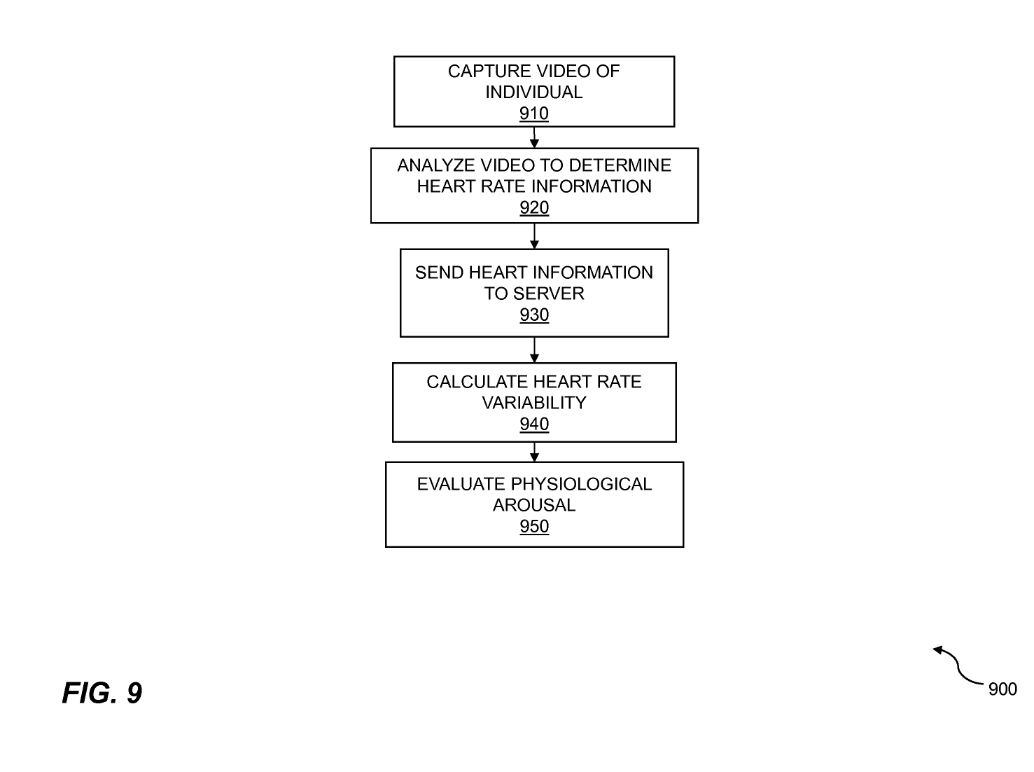

FIG. 9 is a flow for physiology analysis with video. The flow 900 describes a computer-implemented method for physiology analysis from a video. The flow 900 can be considered from the perspective of a video capture machine. The flow 900 can include capturing video of an individual 910. The video capture can be accomplished by any image capture device including a webcam, a phone camera, a tablet camera, a wearable camera, and a room camera. The video capture can be accomplished through a wearable device including a camera which can be used for gathering contextual information and/or collecting heart rate information on the individual or on other users.

The flow 900 can include analyzing the video to determine heart rate information 920. The analysis information can include both video and collected information relating to mental states. The video analysis can infer a heart rate variability. The video analysis can include an algorithm that detects the underlying heartbeat. The video analysis can include analyzing differences in the reflectivity of ambient light on an individual's face, with changes in reflectivity over time representing variations in heart rate. Other information or analysis methods of determining heart rate information can be presented in the video analysis. The video analysis can include other information about the mental state of the individual, including facial data, physiological data, accelerometer data, and analysis of an individual's face to extract and interpret laughs, smiles, frowns, and other facial expressions.

The flow 900 can include sending the heart rate information to a server 930 for further analysis. In some embodiments, the server is the same computer that is analyzing the video. In some embodiments, the server is a different computer than the computer that is analyzing the video. In some embodiments, the server computer is linked to other computers, including the computer analyzing the video via the Internet or another computer network.

The information can include an evaluation of data including the heart's pulse wave. The information can also include an analysis of the skin color change as a function of blood volume changes. Other methods to extract heart rate data can also be applied when analyzing a video to determine heart rate information. The heart rate information can include information which exhibits an increase in heart rate derived from measuring the sympathetic nervous system, and the heart rate information can include information which exhibits a decrease in heart rate derived from measuring the parasympathetic or vagal nervous system. In some embodiments, the data sent to the server can include other mental state data such as, but not limited to, facial data, respiration rate, blood pressure, skin resistance, skin temperature, accelerometer data, mental state inference, audible sounds, gestures, electrodermal data, and/or contextual data. In some embodiments, the data sent to the server is a subset of the data which was captured on the individual.

The server analysis can include calculating a heart rate variability value 940 based on the heart rate information. The server can analyze heart rate information that includes low frequency heart rate values determined from the heart rate information. The low frequency heart rate values can be based on measurements between 0.04 Hz to 0.15 Hz. The server can analyze heart rate information that includes high frequency heart rate values determined from the heart rate information. The high frequency heart rate values can be based on measurements between 0.15 Hz to 0.4 Hz. The analysis can include calculating a heart rate variability value based on the low frequency heart rate value and the high frequency heart rate value. The heart rate variability can also be inferred.

Based on the heart rate variability value, physiological arousal can be evaluated 950. The physiological arousal can include an emotional response. The physiological arousal can be used in media analysis where multiple media instances have heart rate variability values correlated to the emotional response for the multiple media instances. In some cases, a variation in heart rate can be correlated to a video or other media that a viewer is experiencing. In some embodiments, the physiological arousal can be correlated to the emotional content of media. Various steps in the flow 900 may be changed in order, repeated, omitted, or the like without departing from the disclosed concepts. Various embodiments of the flow 900 may be included in a computer program product embodied in a non-transitory computer readable medium that includes code executable by one or more processors.

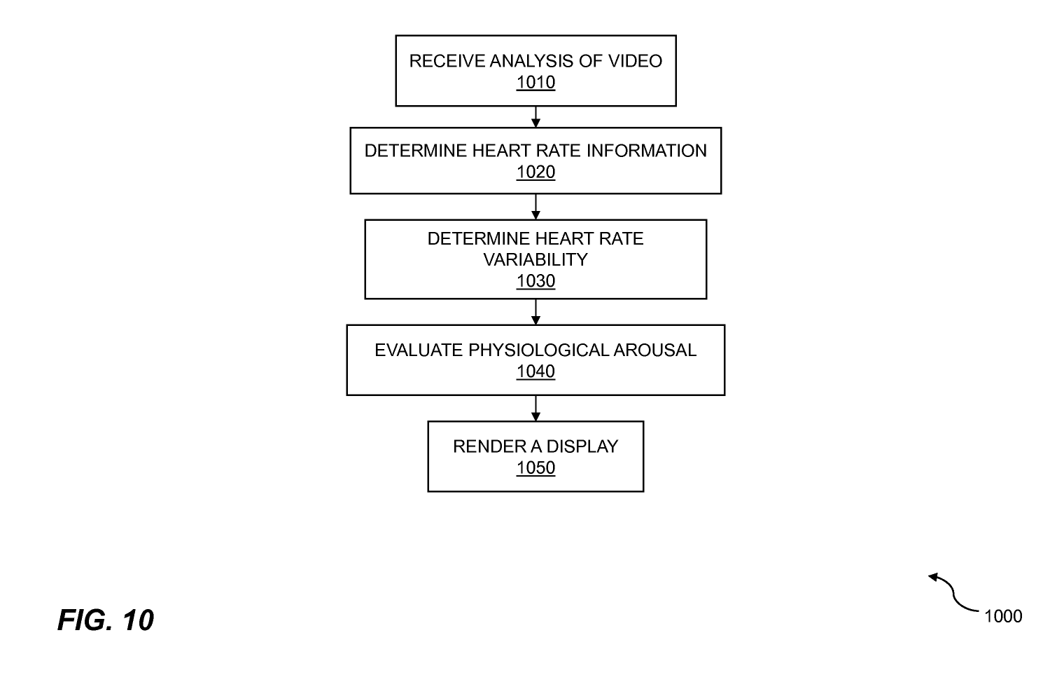

FIG. 10 is a flow for physiology analysis with a server. The flow 1000 describes a computer-implemented method for physiology analysis from the perspective of a rendering machine. The flow 1000 includes receiving analysis of a video 1010. The video analysis can infer a heart rate variability. The analysis information can include both video and collected information relating to mental states. The video analysis can include an algorithm that detects the underlying heartbeat. Other information or analysis methods for determining heart rate information can be presented in the video analysis. The video analysis can include other information about the mental state of the individual, including facial data, physiological data, accelerometer data, and analysis of an individual's face to extract and interpret laughs, smiles, frowns, and other facial expressions.

The flow 1000 includes analyzing the video to determine heart rate information 1020. The analysis can include an evaluation of data including the heart's pulse wave. The evaluation can also include an analysis of the skin color change as a function of changes in blood volume. Other methods to extract heart rate information can also be applied when analyzing a video to determine heart rate information. In certain embodiments, the analysis can include determining the context, circumstances, and setting under which the video was recorded.

The flow further includes determining a heart rate variability value 1030 based on the heart rate information. The server can analyze heart rate information that includes low frequency heart rate values determined from the heart rate information. The low frequency heart rate value can be based on measurements between 0.04 Hz to 0.15 Hz. The server can analyze heart rate information that includes high frequency heart rate values determined from the heart rate information. The high frequency heart rate values can be based on measurements between 0.15 Hz to 0.4 Hz. The analysis can include calculating a heart rate variability value based on the low frequency heart rate value and the high frequency heart rate value. The heart rate variability can be inferred. The heart rate variability value can be based upon the sympathetic and parasympathetic components of the heart rate. The heart rate variability value can be based upon the heart rate ratio. The heart rate information used to create the value can include mental state data such as data from facial analysis, biosensors, physiological data, and the like.

The flow 1000 includes evaluating physiological arousal 1040 based on the heart rate variability value. A measured scale of arousal can range from a high value, such as when someone is agitated, to a low value, such as when someone is bored. The evaluation can include an assessment of the degree of emotional content associated with the video analysis. The evaluation can include the context under which the evaluation is made. The evaluation can be undertaken for a point in time or over a period of time. The physiological arousal can include an emotional response. The physiological arousal can be used in media analysis where multiple media instances have heart rate variability values correlated to the emotional response for the multiple media instances. In some embodiments, a variation in heart rate can be correlated to a video or other media that a viewer is experiencing. In some embodiments, the physiological arousal can be correlated to the emotional content of media. The analyzing of the heart rate information can be performed by a web service. In some embodiments, the analyzing of the heart rate information can be performed on a mobile device.

The flow 1000 includes rendering a display 1050 of the physiological arousal. The display can be on a laptop computer, a tablet computer, a tablet, mobile phone, a desktop computer monitor, a television, any other type of electronic device, and the like. The physiological arousal can be displayed as a graph, chart, image, text, video, web page, projection, or any other means to represent information. In some embodiments, the rendering of physiological arousal status can occur on a different computer than the computer that is capturing video for heart rate variability analysis or the computer that is analyzing the heart rate information to determine arousal or an individual's mental state. Various steps in the flow 1000 may be changed in order, repeated, omitted, or the like without departing from the disclosed concepts. Various embodiments of the flow 1000 may be included in a computer program product embodied in a non-transitory computer readable medium that includes code executable by one or more processors.

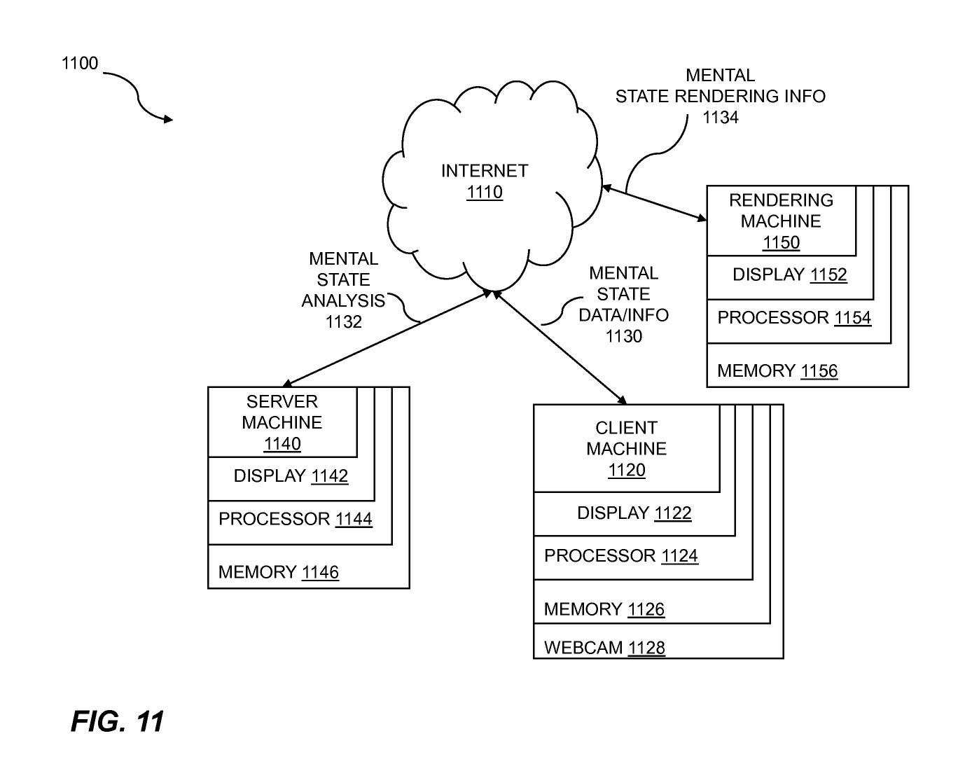

FIG. 11 is an example of a system for heart rate variability analysis usage. The system 1100 can include a computer program product embodied in a non-transitory computer readable medium for mental state analysis, the computer program product comprising: code for obtaining video of an individual; code for analyzing the video to determine heart rate information; code for calculating a heart rate variability value (HRV) based on the heart rate information; and code for evaluating a physiological arousal based on the HRV. The heart rate variability can be based on sympathovagal balance, which is a ratio of a low frequency heart rate value to a high frequency heart rate value. In embodiments, the computer system for performing heart rate variability evaluation for mental state analysis comprises a client machine 1120 configured to collect video images of an individual. The heart rate information collection client machine 1120 can comprise one or more processors 1124 coupled to a display 1122 and a webcam 1128. The display 1122 can be any electronic display, including but not limited to, a computer display, a laptop screen, a net-book screen, a tablet computer screen, a cell phone display, a mobile device display, a remote with a display, a television, a projector, and the like. The webcam 1128, as the term is used herein, can refer to a video camera, a still camera, a thermal imager, a CCD device, a phone camera, a three-dimensional camera, a depth camera, multiple webcams used to show different views of a person, or any other type of image capture apparatus that can allow data captured to be used in an electronic system. The image collection machine can be configured to transmit heart rate information and/or mental state data or information 1130 to a server via the Internet 1110 or other network. The display and the camera can be coupled to a set-top box type device.

An analysis server machine 1140 can obtain heart rate information 1130 from the Internet or other sources. The analysis server machine 1140 can comprise one or more processors 1144 coupled to a display 1142 and a memory 1146 designed to store system information, instructions, and the like. The display 1142 can be any electronic display, including but not limited to, a computer display, a laptop screen, a net-book screen, a tablet computer screen, a cell phone display, a mobile device display, a remote with a display, a television, a projector, or the like. The one or more processors 1144, when executing the instructions which are stored, can be configured to obtain heart rate information and use the information to calculate heart rate variability and/or physiological arousal. The heart rate information and mental state analysis 1132 can be sent via the Internet 1110 to another server, computer, web service or the like.

In some embodiments, the rendering of emotional status can occur on a different computer than the client machine 1120 or the analysis server 1140. The computer can be a rendering machine 1150 which receives heart rate information, mental state information, and/or mental state rendering information 1134 from the client machine 1120, analysis machine 1140, or both. In embodiments, the rendering machine 1150 includes one or more processors 1154 coupled to a memory 1156, and a display 1152. In at least one embodiment, the heart rate information collection machine function, the analysis server function, and/or the rendering machine function are performed by one machine. The system 1100 can include a computer program product comprising code for collecting a video and/or heart rate information, code for evaluating a video, code for evaluating facial images, biometric data, or other information from the individual, code for evaluating heart rate information and/or heart rate variations, code for comparing results from the evaluating of the heart rate information and/or heart rate variability analysis, and code for relating the information to states of arousal for a particular media, such as an advertisement.

Each of the above methods may be executed on one or more processors on one or more computer systems. Embodiments may include various forms of distributed computing, client/server computing, and cloud based computing. Further, it will be understood that the depicted steps or boxes contained in this disclosure's flow charts are solely illustrative and explanatory. The steps may be modified, omitted, repeated, or re-ordered without departing from the scope of this disclosure. Further, each step may contain one or more sub-steps. While the foregoing drawings and description set forth functional aspects of the disclosed systems, no particular implementation or arrangement of software and/or hardware should be inferred from these descriptions unless explicitly stated or otherwise clear from the context. All such arrangements of software and/or hardware are intended to fall within the scope of this disclosure.

The block diagrams and flowchart illustrations depict methods, apparatus, systems, and computer program products. The elements and combinations of elements in the block diagrams and flow diagrams, show functions, steps, or groups of steps of the methods, apparatus, systems, computer program products and/or computer-implemented methods. Any and all such functions—generally referred to herein as a “circuit,” “module,” or “system”—may be implemented by computer program instructions, by special-purpose hardware-based computer systems, by combinations of special purpose hardware and computer instructions, by combinations of general purpose hardware and computer instructions, and so on.

A programmable apparatus which executes any of the above mentioned computer program products or computer-implemented methods may include one or more microprocessors, microcontrollers, embedded microcontrollers, programmable digital signal processors, programmable devices, programmable gate arrays, programmable array logic, memory devices, application specific integrated circuits, or the like. Each may be suitably employed or configured to process computer program instructions, execute computer logic, store computer data, and so on.

It will be understood that a computer may include a computer program product from a computer-readable storage medium and that this medium may be internal or external, removable and replaceable, or fixed. In addition, a computer may include a Basic Input/Output System (BIOS), firmware, an operating system, a database, or the like that may include, interface with, or support the software and hardware described herein.

Embodiments of the present invention are neither limited to conventional computer applications nor the programmable apparatus that run them. To illustrate: the embodiments of the presently claimed invention could include an optical computer, quantum computer, analog computer, mobile device, tablet, wearable computer or the like. A computer program may be loaded onto a computer to produce a particular machine that may perform any and all of the depicted functions. This particular machine provides a means for carrying out any and all of the depicted functions.

Any combination of one or more computer readable media may be utilized including but not limited to: a non-transitory computer readable medium for storage; an electronic, magnetic, optical, electromagnetic, infrared, or semiconductor computer readable storage medium or any suitable combination of the foregoing; a portable computer diskette; a hard disk; a random access memory (RAM); a read-only memory (ROM), an erasable programmable read-only memory (EPROM, Flash, MRAM, FeRAM, or phase change memory); an optical fiber; a portable compact disc; an optical storage device; a magnetic storage device; or any suitable combination of the foregoing. In the context of this document, a computer readable storage medium may be any tangible medium that can contain or store a program for use by or in connection with an instruction execution system, apparatus, or device.

It will be appreciated that computer program instructions may include computer executable code. A variety of languages for expressing computer program instructions may include without limitation C, C++, Java, JavaScript™, ActionScript™, assembly language, Lisp, Perl, Tcl, Python, Ruby, hardware description languages, database programming languages, functional programming languages, imperative programming languages, and so on. In embodiments, computer program instructions may be stored, compiled, or interpreted to run on a computer, a programmable data processing apparatus, a heterogeneous combination of processors or processor architectures, and so on. Without limitation, embodiments of the present invention may take the form of web-based computer software, which includes client/server software, software-as-a-service, peer-to-peer software, or the like.

In embodiments, a computer may enable execution of computer program instructions including multiple programs or threads. The multiple programs or threads may be processed approximately simultaneously to enhance utilization of the processor and to facilitate substantially simultaneous functions. By way of implementation, any and all methods, program codes, program instructions, and the like described herein may be implemented in one or more threads which may in turn spawn other threads, which may themselves have priorities associated with them. In some embodiments, a computer may process these threads based on priority or other order.

Unless explicitly stated or otherwise clear from the context, the verbs “execute” and “process” may be used interchangeably to indicate execute, process, interpret, compile, assemble, link, load, or a combination of the foregoing. Therefore, embodiments that execute or process computer program instructions, computer-executable code, or the like may act upon the instructions or code in any and all of the ways described. Further, the method steps shown are intended to include any suitable method of causing one or more parties or entities to perform the steps. The parties performing a step, or portion of a step, need not be located within a particular geographic location or country boundary. For instance, if an entity located within the United States causes a method step, or portion thereof, to be performed outside of the United States then the method is considered to be performed in the United States by virtue of the causal entity.

While the invention has been disclosed in connection with preferred embodiments shown and described in detail, various modifications and improvements thereon will become apparent to those skilled in the art. Accordingly, the forgoing examples should not limit the spirit and scope of the present invention; rather it should be understood in the broadest sense allowable by law.

What is claimed is:

1. A computer-implemented method for mental state analysis comprising:
obtaining video of an individual;
analyzing the video to determine heart rate information;
calculating a heart rate variability value (HRV) based on the heart rate information; and
evaluating a physiological arousal based on the HRV.

2. The method of claim 1 wherein the physiological arousal includes an emotional response.

3. The method of claim 1 wherein the HRV is based on a low frequency heart rate value determined from the heart rate information.

4. The method of claim 3 wherein the HRV is based on sympathovagal balance.

5. The method of claim 3 wherein the low frequency heart rate value is based on 0.04 Hz to 0.15 Hz.

6. The method of claim 3 wherein the HRV is based on a high frequency heart rate value determined from the heart rate information.

7. The method of claim 6 wherein the high frequency heart rate value is based on 0.15 Hz to 0.4 Hz.

8. The method of claim 6 further comprising calculating a heart rate ratio based on the low frequency heart rate value and the high frequency heart rate value.

9. The method of claim 8 further comprising correlating the heart rate ratio to the physiological arousal.

10. The method of claim 1 wherein:
the physiological arousal includes an emotional response;
the HRV is based on sympathovagal balance where the sympathovagal balance is determined based on a ratio of a low frequency heart rate sympathetic value to a high frequency heart rate parasympathetic value;
the calculating is performed using a sliding time window; and
the physiological arousal is used in media analysis where multiple media instances have HRV values correlated to the emotional response for the multiple media instances.

11. (canceled)

12. The method of claim 1 wherein the calculating is performed using a sliding time window.

13. The method of claim 1 wherein the physiological arousal is used in media analysis.

14. The method of claim 1 wherein the heart rate information is augmented by one or more biosensors.

15. The method of claim 1 further comprising inferring mental states based on the HRV.

16. The method of claim 15 where the mental states include one or more of stress, sadness, happiness, anger, frustration, confusion, disappointment, hesitation, cognitive overload, focusing, engagement, attention, boredom, exploration, confidence, trust, delight, disgust, skepticism, doubt, satisfaction, excitement, laughter, calmness, and curiosity.

17. (canceled)

18. The method of claim 1 wherein the heart rate information is obtained from multiple sources.

19. The method of claim 18 wherein at least one of the multiple sources is a mobile device.

20. The method of claim 18 wherein at least one of the multiple sources is a wearable device.

21. The method of claim 1 wherein the heart rate information is collected sporadically.

22. The method of claim 1 wherein the analyzing of the heart rate information is performed by a web service.

23. The method of claim 1 wherein the analyzing of the heart rate information is performed on a mobile device.

24. The method of claim 1 further comprising determining context during which the heart rate information is captured.

25. A computer program product embodied in a non-transitory computer readable medium for mental state analysis, the computer program product comprising:
code for obtaining video of an individual;
code for analyzing the video to determine heart rate information;
code for calculating a heart rate variability value (HRV) based on the heart rate information; and
code for evaluating a physiological arousal based on the HRV.

26. A computer system for mental state analysis comprising:
a memory which stores instructions;
one or more processors coupled to the memory wherein the one or more processors, when executing the instructions which are stored, are configured to:
obtain video of an individual;
analyze the video to determine heart rate information;
calculate a heart rate variability value (HRV) based on the heart rate information; and
evaluate a physiological arousal based on the HRV.

27-29. (canceled)
